(19) United States
(12) Patent Application Publication
(10) Pub. No.: US 2013/0090647 A1
(43) Pub. Date: Apr. 11, 2013

(54) ABLATION CATHETER WITH INSULATED TIP

(71) Applicant: Boston Scientific Scimed, Inc., Maple Grove, MN (US)

(72) Inventor: SCOTT R. SMITH, CHASKA, MN (US)

(73) Assignee: BOSTON SCIENTIFIC SCIMED, INC., MAPLE GROVE, MN (US)

(21) Appl. No.: 13/631,481

(22) Filed: Sep. 28, 2012

Related U.S. Application Data
(60) Provisional application No. 61/545,973, filed on Oct. 11, 2011.

(51) Int. Cl. A61B 18/14

(52) U.S. Cl. 606/41

(57) ABSTRACT

An ablation catheter configured to be navigated through a vessel to ablate tissue, the ablation catheter comprising an elongate catheter shaft having a proximal end and a distal end. An electrode is positioned near the distal end of the elongate shaft, and is configured to transmit radio-frequency energy into a vessel wall. An electrically insulative tip at the distal end of the catheter keeps the electrode away from the blood vessel wall.

### CROSS-REFERENCE TO RELATED APPLICATIONS

This application claims priority under 35 U.S.C. §119 to U.S. Provisional Application Ser. No. 61/545,973, filed Oct. 11, 2011, the entirety of which is incorporated herein by reference.

### TECHNICAL FIELD

This disclosure relates to devices and methods for intravascular neuromodulation. More particularly, the technologies disclosed herein relate to apparatus, systems, and methods for achieving intravascular renal neuromodulation via thermal heating.

### BACKGROUND

Certain treatments require the temporary or permanent interruption or modification of select nerve function. One example of such a treatment is renal nerve ablation, which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels, and thus can be accessed intravascularly through the walls of the blood vessels. In some instances, it may be desirable to ablate perivascular renal nerves using a radio frequency (RF) electrode. However, such a treatment may result in thermal injury to the vessel wall at the electrode, and other undesirable side effects, such as, but not limited to, blood damage, clotting and/or protein fouling of the electrode.

It is therefore desirable to provide for better systems and methods for intravascular nerve modulation.

### SUMMARY

The disclosure is directed to several alternative designs, materials, and methods of manufacturing medical device structures and assemblies for performing nerve ablation.

Accordingly, one illustrative embodiment is an ablation catheter configured to be navigated through a vessel to ablate tissue, the ablation catheter comprising an elongate catheter shaft having a proximal end and a distal end. An electrode is positioned near the distal end of the elongate shaft, and is configured to transmit radio-frequency energy into a vessel wall. An electrically insulative tip at the distal end of the catheter keeps the electrode away from the blood vessel wall.

Some embodiments pertain to a method of ablating perivascular renal nerves, comprising navigating an ablation catheter through a vasculature to a vessel lumen of a vessel, the ablation catheter including an elongate shaft having a tip electrode on a distal end portion of the elongate shaft and an electrically insulating tip distal of the tip electrode. The tip electrode includes an active surface extending proximally of the electrically insulating tip. The method further includes deflecting the distal end portion toward a wall of the vessel to position the electrically insulating tip against the vessel wall, and activating the tip electrode to emit radio-frequency energy from the active surface through the wall of the vessel to nerve tissue. The active surface of the tip electrode is spaced away from the wall of the vessel when the electrically insulating tip is positioned against the vessel wall.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 is a side view of an exemplary embodiment of a distal end of a renal ablation system received in a blood vessel.

FIG. 3 is a side view of a distal end of an illustrative renal ablation system, depicting current and blood flow.

FIG. 4 is a side view of an alternate embodiment of the renal ablation system, shown in FIG. 2.

FIG. 5 is a side view of another embodiment of the renal ablation system, shown in FIG. 2.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification. All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to RF ablation of perivascular renal nerves for treatment of hypertension, it is contemplated that the devices and methods may be used in other applications where nerve modulation and/or ablation are desired.

The present disclosure provides methods and systems to ablate a renal nerve. To this end, the system employs a catheter carrying one or more electrodes at its distal end to ablate renal nerves by passing into the nerves radio frequency energy. The distal portion of the catheter is bent to point towards the target nerve using a known steering mechanism. In the alternative, the catheter may have a pre-formed bent distal portion. In either configuration, an electrically insulative member, such as the distal tip of the catheter contacting the artery walls is insulated to prevent direct contact between the electrode and the vessels walls. The insulated tip or other insulative member acting as a barrier between the electrode and the vessel wall enables the electrode to be spaced apart from the artery walls, avoiding current concentrations at the artery walls, and distributing the ablation energy uniformly across the target nerve. Further, positioning the electrode away from the vessel wall provides some degree of passive cooling by allowing blood to flow past the electrode.

### EXEMPLARY EMBODIMENTS

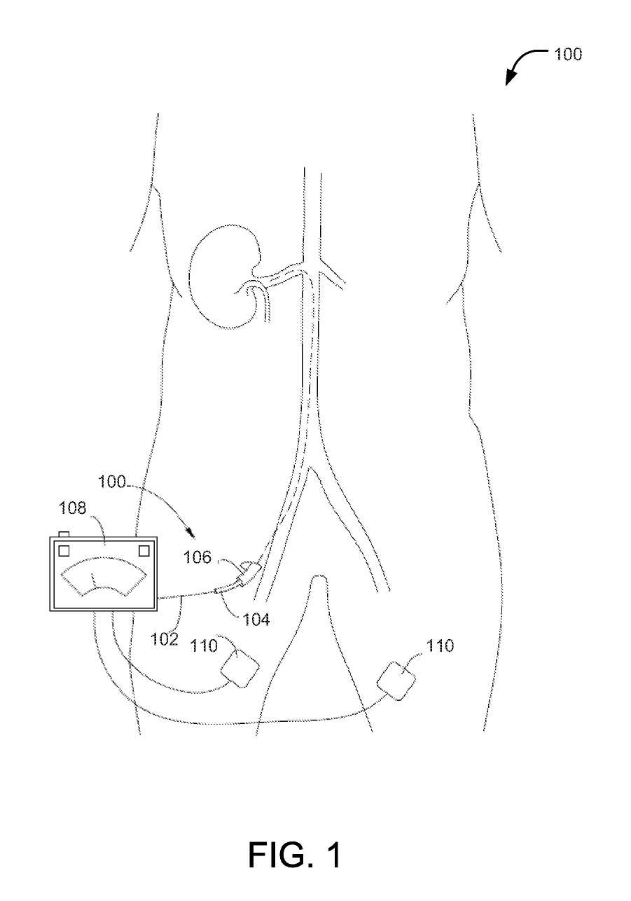

FIG. 1 is a schematic view of an illustrative renal nerve modulation system 100 in situ. System 100 may include one or more conductive element(s) 102 providing power to renal ablation system 104 disposed within a sheath 106, the details of which can be better seen in subsequent figures.

A proximal end of conductive element 102 may be connected to a control and power element 108, which supplies the necessary electrical energy to activate the one or more electrodes at or near a distal end of the renal ablation system 104. In some instances, return electrode patches 110 may be supplied on the legs or at another conventional location on the patient's body to complete the circuit. The control and power element 108 may include monitoring elements to monitor parameters such as power, temperature, voltage, pulse size and/or shape and other suitable parameters as well as suitable controls for performing the desired procedure. The power element 108 may control a radio frequency (RF) electrode, which may be configured to operate at a frequency of approximately 460 kHz, for example. It is contemplated that any desired frequency in the RF range may be used, for example, from 450-500 kHz. It is, however, contemplated that different types of energy outside the RF spectrum may be used as desired, for example, but not limited to ultrasound, microwave, and laser.

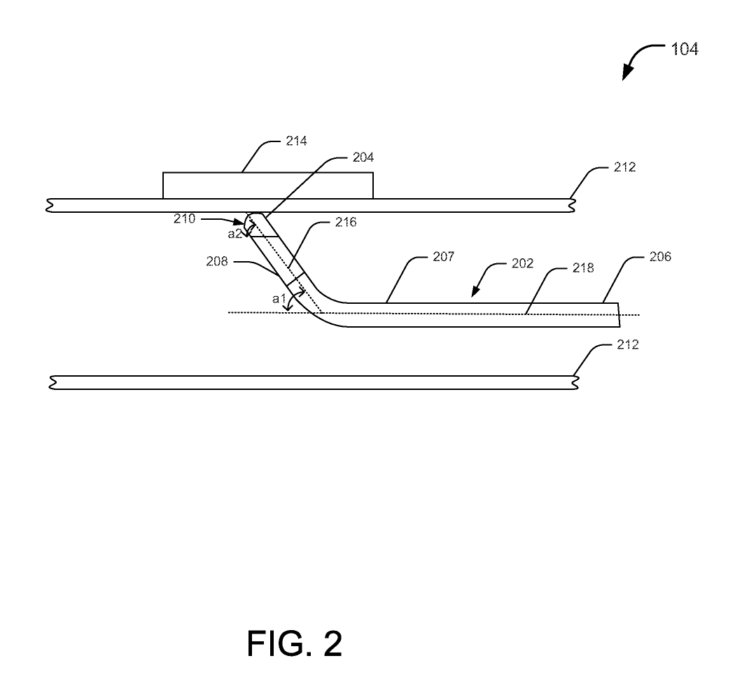

FIG. 2 illustrates a side view of an exemplary embodiment of a distal end of the renal ablation system 104. The renal ablation system 104 may include an elongated catheter 202 having a proximal end 206, a distal end 204, and an elongated shaft 207 extending from the proximal end 206 to the distal end 204. The distal end 204 may further include an electrode 208 for transmitting ablation energy to the desired body tissue. In addition, an electrically insulated material may form an insulated tip 210 at the distal tip of the electrode 208, or another electrically insulative member acting as a barrier member.

Catheter 202 may be adapted to advance into a body lumen having a vessel wall 212 to ablate a body tissue 214. Catheter 202 may be hollow, with a cross-sectional configuration adapted to be received in a desired body lumen, such as a renal artery. In the illustrated embodiment, catheter 202 may be generally circular, with a generally circular hollow interior lumen. Further, the catheter 202 may have a uniform diameter, but in other embodiments (not shown), the catheter 202 may taper at its distal end 204 to allow convenient insertion into the body. In addition, depending upon the particular implementation and intended use, the length of catheter 202 may vary. For instance, the catheter 202 may have a sufficient length such that the distal end 204 may extend into the body lumen while the proximal end 206 remains outside of a patient's body. The catheter 202 may further include one or more lumens configured in a number of ways in the art. For example, the elongated shaft 207 may include a guidewire lumen, which may extend completely or partially along the entire length of the elongated shaft 207 for receiving a guide wire therein.

The distal portion of the catheter 202 may be bent at a desired angle, directing towards the target tissue 214. To this end, in some embodiments the catheter 202 may be fabricated with the distal portion being bent at a predetermined angle such that the distal end portion automatically reverts to the pre-formed bent shape when unconstrained. As shown, the distal portion of catheter 202 may include a longitudinal axis, shown as a dotted line 216. In addition, the proximal portion of the elongate shaft 207 extending proximal of the distal portion may have a central longitudinal axis, shown as a dotted line 218. An angle a1 between the two dotted lines 216 and 218 defines the bent angle of the distal portion. Angle a1 may be an oblique angle, such as 30 degrees, 45 degrees, 60 degrees, or any desired angle, for example. In some instances, the angle a1 may be selected to position the electrode 208 at a desired distance from the vessel wall 212 or other desired orientation and/or permit a desired flow of blood past the electrode 208.

In an alternate embodiment, the catheter 202 may include a steering mechanism (not shown) to manually bend the distal portion at a desired angle once the catheter is positioned close to the tissue 214. For example, pull wires may be connected to the distal end 204 of the catheter 202 and may be contained in a lumen (not shown) of the catheter 202. These pull wires may extend up to the proximal end 206 and can terminate in a slider, for example, which can be manipulated by an operator. In one implementation, the slider can move in a slot, which pulls or pushes the wire. Moving the slider results in bending or unbending of the distal portion as desired.

Angling the distal end portion of the catheter 202 at the angle a1 may reduce and/or prevent flow detachment of the blood, and thus may provide a form of boundary layer control of blood flowing past the catheter shaft 207. In some instances, the angled cylindrical configuration of the distal portion of the catheter 202 with the electrode 208 positioned thereon, may create spiraling blood flow around the electrode 208 to reduce the thickness of the boundary layer of the blood past the electrode 208. Accordingly, the configuration of the angle a1 may permit more efficient heat transfer away from the electrode 208 and/or vessel wall 212.

In one instance (not shown), the proximal end 206 may include a handle portion adapted to hold the catheter 202, while a portion of the catheter 202 is inserted into a patient's body. The handle may include a hub for connecting other treatment devices or providing a port for facilitating other treatments. In addition, the handle or the proximal end of the catheter 202 may be connected to an ablation source, which supplies the necessary electrical energy to activate one or more electrodes at the distal end of the catheter 202. The handle may also include a steering mechanism, such as the slider connected to pull wires, for steering the distal end of the catheter 202. In still other embodiments, other active deflection mechanisms can be used.

Catheter 202 may be made of, for example, a polymeric, electrically nonconductive material, such as polyethylene, polyurethane, or PEBAX® material (polyurethane and nylon). Alternatively, the catheter 202, or a portion thereof, may be made from a malleable material, such as stainless steel or aluminum, allowing a physician to change the shape of the catheter 202 before or during an operation. In some instances, the catheter 202 may be composed of an extrusion of wire braided polymer material to impart flexibility. In addition, the distal end 204 may be made softer than the proximal portion by using different material and/or having a thinner wall thickness. This may have the benefit of reducing the risk of injury to vessel walls, which the distal end 204 may contact, during an operation. The catheter 202 may also be coated using suitable low friction material, such as TEFLON®, polyetheretherketone (PEEK), polyimide, nylon, polyethylene, or other lubricious polymer coatings, to reduce surface friction with the surrounding body tissues.

Electrode 208 may be a single electrode or an array of electrodes connected to each other or individual electrodes that are electrically independent of each other. These electrodes may be disposed on the outer surface of the catheter's distal end. In some embodiments, the electrode 208 may be a separate tubular or cylindrical structure attached to the distal end of the catheter 202. For example, the electrode 208 may be machined or stamped from a monolithic piece of material and subsequently, bonded or otherwise attached to the elongate shaft 207. In other embodiments, the electrode 208 may be formed directly on the surface of the elongate shaft 207. For example, the electrode 208 may be plated, printed, or deposited on the surface. It is contemplated that the electrode 208 may take any shape desired, such as, but not limited to, square, rectangular, circular, or oblong.

In addition, each electrode 208 may be connected by the conductive element 102 to the ablation source at the proximal end of the catheter 202. The ablation source may be used for delivering ablation energy to the electrode 208 to ablate target tissue during use. The ablation source may be a radio frequency (RF) generator or any known source that provides ablation energy to the electrode 208. Each electrode 208 may have a separate electrical connection through the conductive element to the ablation source, or there may be a single conductive element common to each electrode 208.

In use, the catheter 202 may ablate the desired target tissue, such as perivascular renal nerves. As energy passes from the electrode 208, it may heat up the artery walls. Further, as the ablation energy increases, the temperature of the artery wall may increase. Higher temperatures, however, may result in thermal injury to the artery walls. It may be, therefore, desirable to position the electrode 208 off the artery walls (i.e., avoid directly contacting the artery wall 212 with the electrode 208).

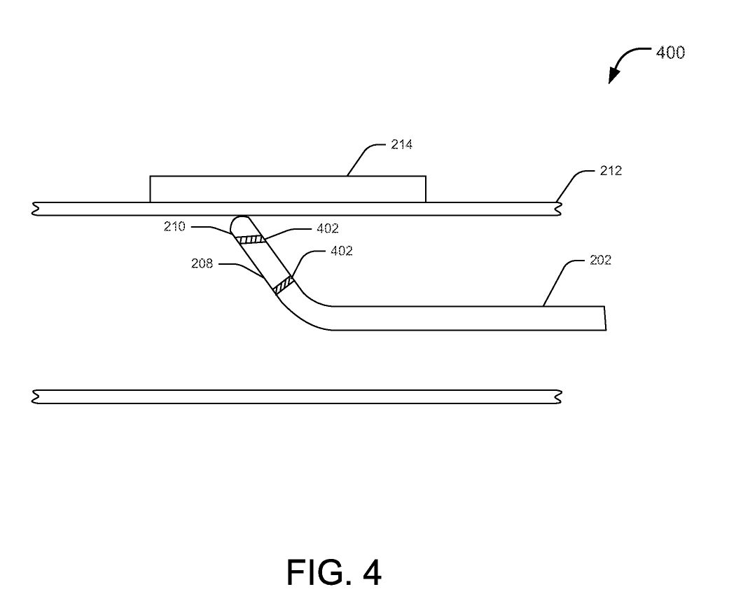

To avoid ablation side effects, the distal tip of the electrode 208 may be electrically insulated to keep the electrode 208 spaced apart and electrically isolated from the artery walls 212. For electrical insulation, a thin layer of an electrically insulative material may be disposed at the distal tip of the electrode 208. In addition, the material forming the tip 210 may be thermally conductive to act as a heat sink, conducting heat away from the vessel walls 212. Suitable materials to manufacture the insulted tip 210 may include a diamond-like carbon (DLC) coating, parylene, a ceramic material (for example, aluminum oxide, aluminum nitride, titanium nitride, sapphire, boron nitride, or beryllium oxide), highly filled polymers (for example, polymers filled with metal or metal oxide), other similar polymers, or other material having similar properties. If heat conduction through the electrode end is not required for increased vessel wall or electrode cooling, a polymer tip at 210, as shown in FIG. 4, can be used, with a simple cylindrical electrode 208 positioned a short distance back from the end of the catheter 202.

In other embodiments, such as embodiments in which a cylindrical electrode 208 is positioned generally parallel to the longitudinal axis of the vessel, an electrically insulative member may be provided along a length of the cylindrical electrode 208 to form an insulative barrier between the cylindrical electrode 208 and the vessel wall. The insulative member may extend for less than the full circumference around the cylindrical electrode 208, leaving a portion of the electrode 208 spaced from the vessel wall exposed. For example, in some instances, the electrically insulative member may be a strip of electrically insulative material extending along one side of the electrode 208.

The insulated tip 210, or other insulated member, may maintain a gap between the artery walls 212 and the exposed electrode 208. The gap or distance between the exposed electrode 208 and the artery wall 212 may allow the current from the electrode 208 to spread out somewhat, reducing the local current density at the vessel wall 212 and placing the active surface of the electrode 208 out in the flowing blood for improved cooling of the electrode 208.

In some embodiments, to maintain a consistent gap between the artery walls 212 and the exposed surface of the electrode 208, the proximal end of the insulated tip 210 may be angled at an oblique angle to the longitudinal axis 216 of the distal bent portion of the catheter 202. As shown, an angle a2 defines the angle between the proximal end of the insulation tip 210 and the longitudinal axis 216 of the distal portion of the catheter 202. In some instances, the angle a2 may be chosen to be substantially the same as the angle a1 of the distal portion of the catheter 202 when deflected or bent into engagement with the vessel wall 212. For example, in some instances, the angle a2 may be about 30 degrees, 45 degrees, 60 degrees, or other angle equivalent to the angle a1. Thus, the proximal end of the insulative tip (and thus the distal end of the exposed portion of the electrode 208) may extend generally parallel to the vessel wall 212 when the distal tip 210 of the catheter 202 is deflected away from the central longitudinal axis 218 of the proximal portion of the catheter shaft 207. Accordingly, the distal extent of the exposed portion of the electrode 208 may be substantially equidistantly oriented from the vessel wall 212 on both a proximal (upstream) and distal (downstream) side of the electrode 208.

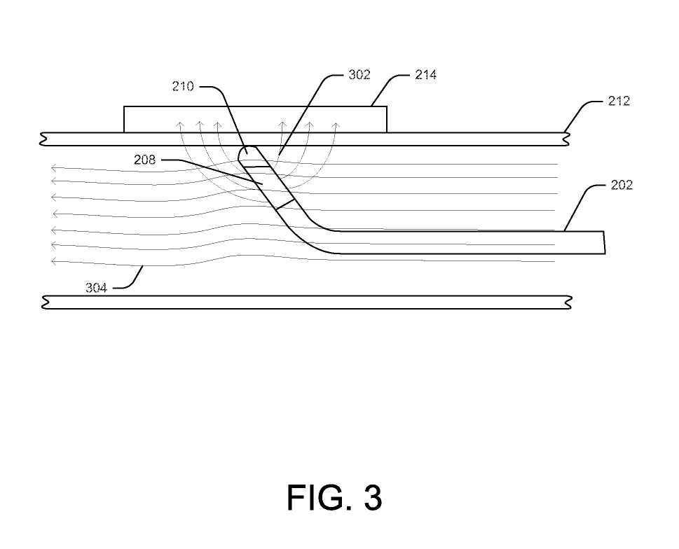

The insulated tip 210 may reduce the risk of the artery walls 212 being directly touched by the electrode 208. FIG. 3 is an embodiment of the distal end of the renal ablation system 104 illustrating an exemplary RF current path and blood flow. As shown, the gap between the exposed electrode 208 may allow the current passing from the electrode 208 to spread out, as shown by dotted lines 302, and traverse through blood before reaching the target tissue. The insulated tip 210 may be configured to avoid RF energy passing directly from the electrode 208 to the artery walls 212, and consequently, may reduce current density at the artery walls 212. It is noted that the current paths from the electrode 208 fan out in all directions according to the impedance of the media. Thus, it may be desirable to maintain a controlled position of the electrode 208 with respect to the vessel wall 212 so that the current density is high in the adjacent wall and low in the opposite wall of the vessel. High current density in the blood may be offset by convective cooling.

Positioning the electrode 208 away from the artery wall 212 may also provide some degree of passive cooling by allowing blood to flow past the entire active surface of the electrode 208, or a portion thereof. Lines 304 depict an exemplary blood flow path within the artery. As shown, the entire exposed surface of the electrode 208 may be in direct contact with the flowing blood. The cooler blood flowing past the electrode 208 may have a cooling effect, drawing heat away from the electrode 208 and/or the vessel wall 212. Further, keeping the exposed electrodes spaced apart from the artery walls 212 may allow blood to contact a greater surface area of the electrode 208. The blood flow may also facilitate the convective cooling of the tissues surrounding the target area, and reduce artery wall thermal injury, blood damage, and/or clotting. In embodiments where the insulted tip is thermally conductive in nature, the insulated tip may also conduct heat away from the artery walls 212 to further cool the artery at the point of contact.

Different alternatives of the ablation system 104 are contemplated. For example, the edges of the exposed electrode 208 may also be insulated, as shown as 402 in FIG. 4. Any suitable material may coat the proximal and/or distal edges of the electrode 208. In one embodiment, an insulation material may be utilized to coat, cover, or mask off the edges of the electrode 208. The insulative coating 402 may prevent electrical current concentration at the edges, resulting in passing more uniform electrical current to the artery walls and subsequently to the target tissue. To avoid current concentrations, the distal edge, the proximal edge, or both the proximal and distal edges may be insulated, as desired.

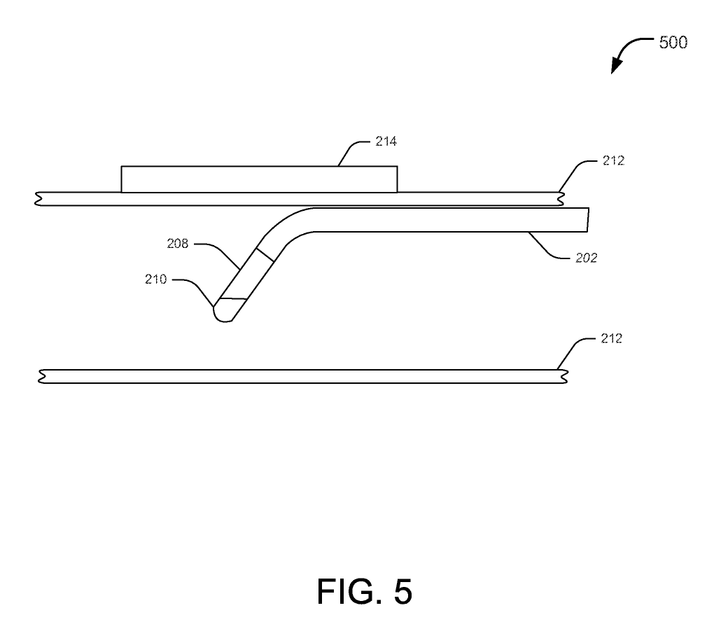

FIG. 5 illustrates an alternate embodiment of the tissue ablation system 500. The system 500 shows an inverse arrangement where the catheter 202 is placed along the artery walls 212 and the deflected distal portion is directed away from the target tissue. In this embodiment, the distal insulated tip 210 may also be spaced apart from the artery walls. The extended gap between the exposed electrode 208 and the artery walls 212 may allow the electrical current to spread out evenly around the target tissue, and prevent the electrode from damaging the artery walls. Furthermore, angling the distal end portion of the catheter 202 at an oblique angle may reduce and/or prevent flow detachment of the blood, and thus may provide a form of boundary layer control of blood flowing past the catheter shaft 207. In some instances, the angled cylindrical configuration of the distal portion of the catheter 202 with the electrode 208 positioned thereon, may create spiraling blood flow around the electrode 208 to reduce the thickness of the boundary layer of the blood past the electrode 208. Accordingly, the configuration of the angle of the distal portion of the catheter 202 may permit more efficient heat transfer away from the electrode 208 and/or vessel wall 212.

In use, the ablation system 104 may assist in ablating the renal nerves. For renal ablation therapy, a physician may advance the ablation system 104 through the vasculature in a manner known in the art. For example, a guide wire may be introduced percutaneously through a femoral artery, and navigated to a renal artery using known techniques such as radiographic techniques. The catheter 202 may then be introduced into the artery over the guide wire until the distal end of the catheter 202 reaches proximal the target tissue.

Subsequently, the physician may manipulate the distal portion of the catheter to point towards the target tissue. In case of a pre-bent catheter 202, the catheter may be introduced enclosed in a sheath (not shown), which constrains the bent distal end into a straightened shape, and once the sheath is withdrawn proximally to extend the distal end portion beyond the sheath, the distal end may automatically bend in its pre-determined state when unconstrained. Alternatively, the ablation system 104 may include an active steering mechanism that may be manually manipulated to bend the distal end towards the target tissue, once deployed. In each configuration, the tip 210 of the catheter 202 may contact the artery walls and the catheter 202 may lie parallel to the artery walls 212, within the center of the artery, as shown in FIGS. 2 and 3.

When the distal end electrode 208 is desirably positioned, radio frequency energy may then be directed from an ablation source to the electrode 208 to ablate the tissue 214, forming a lesion on the contacted tissue. During ablation, the gap maintained between the exposed electrode 208 and the artery walls 212 may allow more uniform distribution of current towards the artery walls. In addition, the blood flow may passively cool the electrode surface in contact therewith. As a result, the present disclosure provides a simple and cost-effective mechanism to ablate a body tissue without damaging surrounding tissues and walls.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. An ablation catheter configured to be navigated through a vessel to ablate tissue, the ablation catheter comprising:
an elongate catheter shaft having a proximal end and a distal end;
an electrode positioned near the distal end of the catheter shaft and configured to transmit radio-frequency energy into a vessel wall; and
an electrically insulative member at the distal end of the catheter shaft that is configured to contact the vessel wall to space the electrode from the vessel wall.

2. The ablation catheter of claim 1, wherein the electrically insulative member is an electrically insulative tip positioned proximate a distal end of the electrode.

3. The ablation catheter of claim 2, wherein the electrically insulative tip is a layer of insulating material covering the distal end of the electrode, and wherein an exposed proximal portion of the electrode is exposed to blood flowing through the vessel.

4. The ablation catheter of claim 3, wherein the exposed proximal portion of the electrode is spaced from the vessel wall.

5. The ablation catheter of claim 4, wherein radio-frequency energy emitted from the electrode passes through blood before reaching the vessel wall.

6. The ablation catheter of claim 2, wherein the electrically insulative tip is a polymer tip at the distal end of the catheter shaft.

7. The ablation catheter of claim 2, wherein the electrically insulative tip is a layer of insulating material covering the distal end of the electrode and wherein an exposed proximal portion of the electrode is exposed to blood flowing through the vessel.

8. The ablation catheter of claim 1, wherein the distal end of the catheter shaft is configured to be deflected towards the vessel wall.

9. The ablation catheter of claim 1, wherein the electrically insulative member is thermally conductive to conduct heat from the vessel wall.

10. The ablation catheter of claim 1, wherein a distal portion of the catheter shaft including the electrode has a central longitudinal axis, wherein a proximal portion of the catheter shaft extending proximal of the distal portion has a central longitudinal axis, and wherein the distal portion is deflected away from the central longitudinal axis of the proximal portion.

11. The ablation catheter of claim 10, wherein the electrically insulative member is an electrically insulative tip having a proximal end covering the distal end of the electrode, the proximal end of the electrically insulative tip being angled at an oblique angle to the central longitudinal axis of the distal portion of the catheter shaft.

12. The ablation catheter of claim 11, wherein the proximal end of the insulative tip extends generally parallel to the vessel wall when the distal portion of the catheter shaft is deflected away from the central longitudinal axis of the proximal portion of the catheter shaft.

13. The ablation catheter of claim 10, wherein the catheter shaft includes a pre-formed fixed bend portion between the distal portion and the proximal portion.

14. An ablation catheter for ablating tissue, the ablation catheter comprising:
an elongate shaft having a proximal end and a distal end;
a tip electrode secured to the distal end of the elongate shaft;
an electrically insulating layer covering a distal most portion of the tip electrode;
wherein the electrically insulating layer blocks radio-frequency energy from passing directly from the tip electrode to a vessel wall when the tip electrode is positioned against the vessel wall.

15. The ablation catheter of claim 14, wherein the tip electrode includes an exposed portion proximal of the electrically insulating layer.

16. The ablation catheter of claim 15, wherein the electrically insulating layer is thermally conductive to conduct heat from the vessel wall.

17. The ablation catheter of claim 15, wherein a proximal end of the electrically insulating layer extends generally parallel to the vessel wall when a distal portion of the elongate shaft is deflected to position the tip electrode against the vessel wall.

18. The ablation catheter of claim 14, wherein the tip electrode is a cylindrical electrode having a proximal edge and a distal edge, wherein the proximal edge and the distal edge are insulated to prevent current concentrations at the proximal and distal edges of the tip electrode.

19. A method of ablating perivascular renal nerves, comprising:
navigating an ablation catheter through a vasculature to a vessel lumen of a vessel, the ablation catheter including an elongate shaft having a tip electrode on a distal end portion of the elongate shaft and an electrically insulating tip distal of the tip electrode, the tip electrode including an active surface extending proximally of the electrically insulating tip;
deflecting the distal end portion toward a wall of the vessel to position the electrically insulating tip against a vessel wall; and
activating the tip electrode to emit radio-frequency energy from the active surface through the vessel wall to nerve tissue;
wherein the active surface of the tip electrode is spaced away from the vessel wall when the electrically insulating tip is positioned against the vessel wall.

20. The method of claim 19, wherein the electrically insulating tip is thermally conductive.
